(19) United States
(12) Patent Application Publication
(10) Pub. No.: US 2009/0233326 A1
(43) Pub. Date: Sep. 17, 2009

(54) METHOD FOR INDUCTION OF THE DIFFERENTIATION OF VISCERAL FAT CELL

(76) Inventors: Jin-ichi Inokuchi, Miyagi (JP); Takashige Sato, Miyagi (JP); Toshio Taira, Hokkaido (JP); Kyoko Shimizu, Hokkaido (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/298,401

(22) PCT Filed: Apr. 23, 2007
(86) PCT No.: PCT/JP2007/058726

§ 371 Date: Oct. 24, 2008

(30) Foreign Application Priority Data
Apr. 24, 2006 (JP) 2006-119749

(51) Int. Cl. C12Q 1/02; C12N 5/06

(52) U.S. Cl. 435/29; 435/377

(57) ABSTRACT

A culture medium is disclosed for inducing the differentiation of visceral preadipocytes into mature visceral adipocytes; the culture medium contains 0.85 to 100 ng/mL insulin and 50 to 250 ng/mL IGF-1. Also disclosed is a method for using the culture medium to induce the differentiation of visceral preadipocytes into mature visceral adipocytes. Use of the differentiation induction system of the present invention enables a substantial induction of adipocyte differentiation without the addition of synthetic differentiation inducers or high insulin concentrations. The mature adipocytes obtained by the differentiation induction system of the present invention are useful for research into the biochemistry and physiology of adipocytes, for screening drugs effective for the treatment of lifestyle-related diseases such as obesity and type 2 diabetes, and for developing diagnostic reagents.

### TECHNICAL FIELD

The present invention relates to a culture medium for culturing visceral adipocytes and inducing the differentiation thereof, a method of culturing visceral adipocytes and inducing the differentiation thereof using the culture medium, and a method of assaying the efficacy of various drug candidates using mature visceral adipocytes obtained by the method of the present invention.

### BACKGROUND

The nutrient components absorbed through the intestinal tract are transported to the liver through the portal vein and lymphatic vessels and are then distributed to tissues throughout the body. There is an ageing-associated increase in the fat tissue in the mesentery where the portal vein and lymphatic vessels are distributed. In recent years it has been evidenced that the excessive accumulation of this mesenteric fat may cause lifestyle-related diseases such as diabetes, hyperlipidemia, arteriosclerosis, and so forth. Thus, an understanding of the properties of visceral adipocytes is of great importance for comprehending the pathological picture of these lifestyle-related diseases. The present inventors have already established, for the first time in the world, a novel culture method that induces the differentiation of preadipocytes (visceral stromal cells: VSC), obtained from rat mesenteric adipose tissue, to mature adipocytes (visceral adipocytes: VAC). Characteristic features of the differentiation induction system are that it does not require, for example, dexamethasone or isobutylmethylxanthine, and that it enables VSC-to-VAC differentiation with only natural fatty acids, vitamins, and insulin (Kyoko SHIMIZU, Masato SAKAI, Mamiko ANDO, Hideyuki CHIJI, Teruo KAWADA, Hitoshi MINEO, Toshio TAIRA, Cell Biol. Int. XX (2006) 1-8).

The insulin concentration in the differentiation system was set at 10 μg/mL, i.e., the same as in conventional differentiation systems for primary cultures. However, this insulin concentration is a high, nonphysiological concentration that is several thousand times that of the in vivo blood concentration (0.5 to 3 ng/mL in the rat). Thus, the conventional differentiation conditions are presumed to cause functional abnormalities, e.g., insulin resistance in mesenteric adipocytes, due to their abnormally high insulin concentrations. In order to apply this culture system to research into metabolic syndrome and particularly to screening for therapeutics for lifestyle-related diseases and to the development of diagnostic reagents for lifestyle-related diseases, an important issue is to bring the insulin concentration as close as possible to the physiological expression levels in vivo.

A number of reports have appeared to date to the effect that insulin and insulin-like growth factor-1 (IGF-1) are important factors in differentiation into adipocytes (Boney, C. M., Moats-Staats, B. M., Stiles, A. D., and D'Ercole, A. J. (1994) Endocrinology 135 (5), 1863-1868; Chen, N. X., Hausman, G. J., and Wright, J. T. (1996) J. Anim. Sci. 74 (10), 2369-2375; Kim, H. S., Richardson, R. L., and Hausman, G. J. (1998) Gen. Comp. Endocrinol 112 (1), 38-45; Gerfault, V., Louveau, I., and Mourot, J. (1999) Gen. Comp. Endocrinol 114 (3), 396-404; Grohmann, M., Sabin, M., Holly, J., Shield, J., Crowne, E., and Stewart, C. (2005) J. Lipid Res 46 (1), 93-103. Epub 2004 Oct. 2016). However, synthetic compounds such as dexamethasone and isobutylmethylxanthine, which are potent differentiation inducers, have been required in order to secure high differentiation efficiencies. These synthetic compounds, while having the ability to forcibly determine the direction of differentiation, differ from the physiological conditions for differentiation. In addition, although insulin is generally used to induce differentiation into adipocytes, the insulin concentration used has in many cases far exceeded the physiological concentration (Mackall, J. C., Student, A. K., Polakis, S. E., and Lane, M. D. (1976), J. Biol. Chem. 251 (20) 6462-6464; Student, A. K., Hsu, R. Y., and Lane, M. D. (1980) J. Biol. Chem. 255 (10), 4745-4750; Doi, H., Masaki, N., Takahashi, H., Komatsu, H., Fujimori, K., and Satomi, S. (2005) Tohoku J. Exp. Med. 207 (3), 209-216).

In addition, it has been reported that the 3T3-L1 adipocyte cell line, which has been widely employed as a adipocyte model, also undergoes differentiation into adipocytes in the presence of IGF-1 instead of insulin (Smith, P. J., Wise, L. S., Berkowitz, R., Wan, C., and Rubin, C. S. (1988) J. Biol. Chem. 263 (19), 9402-9408; Boney, C. M., Gruppuso, P. A., Faris, R. A., and Frackelton, A. R., Jr. (2000) Mol. Endocrinol. 14 (6), 805-813; Sekimoto, H., and Boney, C. M. (2003) Endocrinology 144 (6) 2546-2552; Entingh-Pearsall, A., and Kahn, C. R. (2004) J. Biol. Chem. 279 (36), 38016-38024. Epub 32004 Jul. 38017). On the other hand, in inducing the differentiation of MC3T3-G2/PA6, another adipocyte precursor cell line, into adipocytes, the formation of lipid droplets has been observed in the presence of only dexamethasone and isobutylmethylxanthine even without the addition of insulin or IGF-1 (Morito, S., Yaguchi, K., Imada, M., Tachikawa, C., Nomura, M., Moritani, S., Igarashi, M., Yokogawa, K., and Miyamoto, K. (2005) Biol. Pharm. Bull 28 (11), 2040-2045). Thus, a consensus view of the role of insulin and IGF-1 in the induction of differentiation has not been established and the evaluation of their physiological role has been problematic.

With regard to the induction of cell differentiation for primary culture of adipocytes, it has been reported that not only insulin but also IGF-1 is involved in differentiation into adipocytes; however, it is also reported that these differentiation systems require the simultaneous addition of dexamethasone and isobutylmethylxanthine (Entingh-Pearsall, A., and Kahn, C. R. (2004) J. Biol. Chem. 279 (36), 38016-38024. Epub 32004 Jul. 38017). For example, in mesenchymal cell lines, almost no differentiation into adipocytes was observed with IGF-1 alone, while differentiation into adipocytes occurs with the simultaneous addition of dexamethasone and isobutylmethylxanthine (Scavo, L. M., Karas, M., Murray, M., and Leroith, D. (2004) J. Clin. Endocrinol Metab 89 (7), 3543-3553).

Thus, to date, the roles of insulin and IGF-1 in the induction of adipocyte differentiation have been evaluated using only differentiation induction media that contain potent nonphysiological synthetic differentiation inducers. Differentiation induction conditions have not been established that enable an evaluation of the roles of insulin and IGF-1 under physiological conditions in the absence of the synthetic differentiation inducers. Thus, the research done to date has yet to develop a method of inducing the differentiation of adipocytes under physiological conditions, which has produced confusion in the understanding of the physiological state of adipocytes and has strongly hindered drug screening using adipocytes and the development of diagnostic reagents using adipocytes.

The references cited herein are listed below. The contents of these publications are hereby incorporated by reference in its entirety. None of these publications is admitted to constitute prior art of the invention.

Nonpatent Reference 1: Kyoko SHIMIZU, Masato SAKAI, Mamiko ANDO, Hideyuki CHIJI, Teruo KAWADA, Hitoshi MINEO, Toshio TAIRA, Cell Biol. Int. XX (2006) 1-8
Nonpatent Reference 2: Mackall, J. C., Student, A. K., Polakis, S. E., and Lane, M. D. (1976) J. Biol. Chem. 251 (20), 6462-6464
Nonpatent Reference 3: Student, A. K., Hsu, R. Y., and Lane, M. D. (1980) J. Biol. Chem. 255 (10), 4745-4750
Nonpatent Reference 4: Smith, P. J., Wise, L. S., Berkowitz, R., Wan, C., and Rubin, C. S. (1988) J. Biol. Chem. 263 (19), 9402-9408

### DISCLOSURE OF THE INVENTION

An object of the present invention is to establish a system for inducing differentiation that makes it possible to induce the differentiation of mesenteric preadipocytes into mature adipocytes under near-physiological conditions.

The present inventors have discovered that the differentiation of mesenteric preadipocytes into mature adipocytes can be induced by the use of a culture medium that contains insulin in a particular concentration range and IGF-1 in a particular concentration range.

The present invention provides a culture medium for inducing the differentiation of visceral preadipocytes into mature visceral adipocytes, wherein said culture medium contains 0.85 to 100 ng/mL insulin and 50 to 250 ng/mL IGF-1 and is substantially free from indomethacin, dexamethasone, and IBMX. The insulin concentration in the culture medium according to the present invention is preferably 0.85 to 30 ng/mL and more preferably 1 to 5 ng/mL.

In another aspect, the present invention provides a method of inducing the differentiation of visceral preadipocytes into mature visceral adipocytes, comprising the steps of culturing the visceral preadipocytes in a culture medium according to the present invention as described above.

In yet another aspect, the present invention provides a method of assaying the efficacy of a test substance as an agent for ameliorating insulin resistance, comprising the steps of culturing mature visceral adipocytes obtained by induction in a culture medium containing 0.85 to 5 ng/mL insulin and 50 to 250 ng/mL IGF-1, under conditions that provoke insulin resistance, to allow for inducing insulin resistance model visceral adipocytes; and culturing the aforementioned mature visceral adipocytes and the aforementioned insulin resistance model visceral adipocytes in the presence of the test substance, and comparing the adipocyte function of these two types of cells.

In another aspect, the present invention provides a method of assaying the efficacy of a test substance as an agent for treating hyperinsulinemia, comprising the steps of: comparing the level of insulin receptor expression between mature visceral adipocytes cultured in the presence of the test substance in a culture medium containing 0.85 to 5 ng/mL insulin and 50 to 250 ng/mL IGF-1, and mature visceral adipocytes cultured in the presence of the test substance in a culture medium containing 70 to 100 ng/mL insulin and 50 to 250 ng/mL IGF-1.

In another aspect, the present invention provides a method of assaying the efficacy of a test substance as an agent that stimulates the secretion of adiponectin, comprising the steps of culturing mature visceral adipocytes in the presence of the test substance in a culture medium containing 0.85 to 5 ng/mL insulin and 50 to 250 ng/mL IGF-1; and measuring the amount of adiponectin produced or secreted by the cells.

In another aspect, the present invention provides a method of assaying the efficacy of a test substance as an agent that stimulates the secretion of adiponectin in insulin resistance, comprising the steps of comparing the amount of adiponectin produced or secreted by mature visceral adipocytes cultured in the presence of the test substance in a culture medium containing 0.85 to 5 ng/mL insulin and 50 to 250 ng/mL IGF-1, and the amount of adiponectin produced or secreted by mature visceral adipocytes cultured in the presence of the test substance in a culture medium containing 70 to 100 ng/mL insulin and 50 to 250 ng/mL IGF-1.

Use of the differentiation induction system of the present invention enables a substantial induction of the differentiation of adipocytes without the addition of potent synthetic differentiation inducers (e.g., indomethacin, dexamethasone, isomethylbutylxanthine, PPARγ agonists, and so forth) and without the addition of insulin in the high concentrations that may cause insulin resistance. The mature adipocytes obtained by the differentiation induction system according to the present invention are useful for research into the biochemistry and physiology of adipocytes, for screening for drugs effective for the treatment of lifestyle-related diseases, such as obesity and type 2 diabetes, and for the development of diagnostic reagents.

### BRIEF DESCRIPTION OF THE DRAWINGS

FIG. 1 shows the influence of dexamethasone on VAC function;

FIG. 2A shows the influence of insulin concentration on the induction of VSC-to-VAC differentiation;

FIG. 2B shows the influence of insulin concentration on the induction of VSC-to-VAC differentiation;

FIG. 2C shows the influence of insulin concentration on the induction of VSC-to-VAC differentiation;

FIG. 2D shows the influence of insulin concentration on the induction of VSC-to-VAC differentiation;

FIG. 3 shows the influence of insulin concentration on the level of adiponectin expression in VACS;

FIG. 4 shows the level of insulin receptor expression in the course of VAC maturation process;

FIG. 5 shows the influence of IGF-1 concentration on the induction of VSC-to-VAC differentiation;

FIG. 6 shows the induction of VSC-to-VAC differentiation by various combinations of IGF-1 and insulin;

FIG. 7 shows the induction of VSC-to-VAC differentiation by various combinations of IGF-1 and insulin;

FIG. 8 shows the insulin receptor expression when VAC differentiation is induced by insulin at a high concentration;

FIG. 9 shows the influence of the insulin concentration on the amount of adiponectin production in the presence of 200 ng/mL IGF-1; and

FIG. 10 shows the influence of troglitazone on insulin receptor expression.

### DETAILED DESCRIPTION OF THE INVENTION

The present invention provides new findings with regard to the procedure for inducing the differentiation of visceral preadipocytes under physiological conditions. To date, the methods generally used to induce adipocyte differentiation have employed high insulin concentrations that are several thousand times the physiological concentration. Moreover, there has been no research where a detailed examination was made as to what insulin concentrations are required at what time periods in the differentiation process. Accordingly, in the present invention, preadipocytes were first cultured in the presence of different insulin concentrations in order to examine the influence of the insulin concentration and insulin treatment schedule on adipocyte differentiation and maturation. The following was elucidated as a result (Example 3). 1) The induction of VSC differentiation in the initial three-day period was achieved with 10 μg/mL insulin, but was not achieved with 0.2 μg/mL insulin. Given that an insulin concentration of 0.2 μg/mL is a concentration nearly 100 times higher than that of the physiological concentration, the presence of another physiological factor in the induction of initial VSC differentiation was strongly suggested. 2) On the other hand, the VAC maturation process after the induction of initial VSC differentiation required insulin at approximately 10 times the physiological concentration. 3) When a high insulin concentration was present after the induction of initial VSC differentiation, expression of the insulin receptor was reduced and insulin resistance was induced.

Then the possibility that other differentiation-inducing factors contribute to differentiation into VACs was examined. It was found that initial differentiation is induced by the presence of a physiological concentration of IGF-1 (Example 4). Furthermore, it was also found that, while IGF-1 alone sufficiently functions in the induction of initial differentiation, both IGF-1 and insulin are required in the maturation process, indicating that insulin in a physiological concentration promotes the formation of lipid droplets concomitantly with IGF-1 (Example 5). That is, the presence of both IGF-1 and insulin in combination resulted in successful differentiation and maturation into adipocytes within a physiological concentration range. It was found that the mature adipocytes obtained in this manner exhibit normal adipocyte function with regard to, for example, lipid droplet formation, insulin receptor expression, adiponectin production, and so forth.

The present invention provides a culture medium for inducing the differentiation of visceral preadipocytes into mature visceral adipocytes, wherein said culture medium contains insulin in a particular concentration range and IGF-1 in a particular concentration range and is substantially free from indomethacin, dexamethasone, or IBMX. The insulin and IGF-1 concentrations in the culture medium of the present invention are near the in vivo biological concentrations for healthy humans and for disease-afflicted humans. The insulin concentration in the culture medium of the present invention is 0.85 to 100 ng/mL, preferably 0.85 to 30 ng/mL, and more preferably in the physiological concentration range for healthy humans (0.85 to 5 ng/mL), particularly preferably about 0.85 ng/mL. As described below, when the present invention is applied to a hyperinsulinemia model system, the insulin concentration may be 50 to 100 ng/mL, more preferably 60 to 80 ng/mL, and particularly preferably approximately 70 ng/mL. The IGF-1 concentration in the culture medium of the present invention is 50 to 250 ng/mL, preferably 190 to 210 ng/mL, and more preferably approximately 200 ng/mL. For example, mature adipocytes that exhibit normal function can be obtained by exposing visceral preadipocytes to a medium containing 200 ng/mL IGF-1 and 0.85 ng/mL insulin.

The culture medium of the present invention preferably contains little or no indomethacin, dexamethasone, IBMX, or a PPAR-γ agonist, which are differentiation inducers employed in the conventional methods used for inducing the differentiation of adipocytes. This is because the presence of these substances in the culture medium may interfere with research into the process of adipocyte differentiation, as well as research into the effects of differentiation inhibitors and differentiation inducers.

The differentiation induction system according to the present invention contains little or no synthetic chemical differentiation inducers such as dexamethasone and IBMX employed in the conventional methods used for inducing adipocyte differentiation, which enables preparation of mature adipocytes that exhibit normal function expressing adipocyte differentiation markers C/EBPα and aP2, without disruption of insulin receptor expression and adiponectin production.

In another aspect, the present invention provides a method of inducing the differentiation of visceral preadipocytes into mature visceral adipocytes, comprising the steps of culturing the visceral preadipocytes in a culture medium according to the present invention as described above.

The method of the present invention for inducing the differentiation of adipocytes is carried out under near-physiological conditions, and, as a consequence, the mature adipocytes yielded by the method are useful in the various types of research that employ adipocytes. For example, they are useful in research into adipocyte metabolism and function (e.g., fat uptake and release, adiponectin production and secretion, insulin receptor expression, and so forth), in the elucidation of the physiological mechanisms of adipocyte differentiation and maturation maintenance in adipocytes, and in the elucidation of signal transduction mechanisms in the processes of adipocyte differentiation and maturation in the physiological conditions.

In another aspect, the present invention provides a method of determining the efficacies of agents for ameliorating insulin resistance, adiponectin secretion promoters, insulin mimics, and so forth, using mature adipocytes with normal functions, as well as a method of screening drug candidates from diverse test substances.

The test substance can be obtained from various libraries such as synthetic compound libraries, natural compound libraries, combinatorial libraries, oligonucleotide libraries, peptide libraries, and so forth. Extracts from natural materials (e.g., bacteria, fungi, seaweed, plants, animals, and so forth) and partially purified materials therefrom may also be used as the test substance.

One embodiment of the methods for determining efficacy of drugs and screening is a method of assaying the efficacy of a test substance as an agent that ameliorates insulin resistance. This method can be carried out by obtaining mature visceral adipocytes by induction with the culture medium of the present invention as described above; culturing said mature visceral adipocytes under conditions that cause insulin resistance to obtain insulin resistance model visceral adipocytes; culturing the mature visceral adipocytes and insulin resistance model visceral adipocytes in the presence of the test substance; and comparing the adipocyte function of the two cell types.

Exemplary conditions capable of inducing insulin resistance may include the addition of fatty acid, glucosamine, or an inflammatory cytokine (TNFa, IL-1, IL-6, and so forth) to the physiological differentiation induction culture medium of the present invention. Insulin receptor expression, adiponectin production, the glucose uptake capacity, and so forth, are examples of the adipocyte function that can be employed as an indicator for searching for and determining the efficacies of agents that may ameliorate insulin resistance. Substances that are candidate agents for ameliorating insulin resistance can be identified by comparing the adipocyte functions of the insulin resistance model visceral adipocytes with those of normal-functional mature adipocytes obtained in accordance with the present invention.

An example of the method for evaluating candidate drug substances is provided below. The efficacy of a candidate anti-hyperlipidemic agent on adiponectin secretion by VACs can be examined using VACs obtained from the differentiation induction system of the present invention. Compounds that have an inhibitory activity on HMG-COA reductase are used as the test compounds. VACs are cultured for five days with 200 ng/mL IGF-1 and 2.5 ng/mL insulin; each test compound is added and continued culturing for four days under the same conditions; and then the amount of adiponectin present in the culture medium is measured. If a test compound inhibits adiponectin secretion, that compound has the potential of having a selective toxicity on adipocytes in vivo. In this manner, a determination of the efficacy of a drug as well as safety screening may be carried out using the physiological differentiation conditions for VACs of the present invention.

Moreover, as shown in Example 7 below, it was found in the present invention that adiponectin secretion is inhibited by culture with insulin at 70 ng/mL or more and that adiponectin secretion is also inhibited by exposure during the maturation process to insulin at 70 ng/mL or more. That is, in another embodiment of the present methods for determining drug efficacies and screening, the efficacy of a test substance as an agent for treating hyperinsulinemia is assayed. This method is carried out by comparing the level of insulin receptor expression between mature visceral adipocytes cultured in the presence of the test substance in a culture medium containing 0.85 to 5 ng/mL insulin and 50 to 250 ng/mL IGF-1 and mature visceral adipocytes cultured in the presence of the test substance in a culture medium containing 70 to 100 ng/mL insulin and 50 to 250 ng/mL IGF-1. For example, if a test substance can inhibit the decline in the level of insulin receptor expression in mature visceral adipocytes cultured in the differentiation induction medium with 70 ng/mL insulin as a model of high plasma insulin levels (an initial pathological change in type 2 diabetes), such a substance may be regarded as a candidate agent for treating hyperinsulinemia.

In another embodiment of the methods for determining and screening drug efficacy, the efficacy of a test substance as an agent that stimulates the secretion of adiponectin is assayed. This method can be carried out by culturing mature visceral adipocytes in the presence of the test substance in the culture medium according to the present invention and measuring the amount of adiponectin produced or secreted by the cells. A substance that can inhibit adiponectin secretion may be regarded as a candidate agent for ameliorating insulin resistance. Conversely, if a test substance inhibits adiponectin secretion, this compound has the potential of having a selective toxicity on adipocytes in vivo.

In another embodiment of the methods for determining drug efficacy and screening, the efficacy of a test substance as an agent that stimulates adiponectin secretion in insulin-resistant conditions is assayed. In the method, the amount of adiponectin produced or secreted by mature visceral adipocytes cultured in the presence of the test substance in a culture medium containing 0.85 to 5 ng/mL insulin and 50 to 250 ng/mL IGF-1 is compared with those of mature visceral adipocytes cultured in the presence of the test substance in a culture medium containing 70 to 100 ng/mL insulin and 50 to 250 ng/mL IGF-1. In addition, the efficacy of a test substance as an agent that stimulates adiponectin secretion in insulin resistance may be assayed by applying the candidate substance to mature adipocytes that exhibit reduced adiponectin production as obtained under nonphysiological conditions, for example, in the presence of both 200 ng/mL IGF-1 and 70 ng/mL insulin, and measuring adiponectin production or secretion.

The culture medium according to the present invention for inducing the differentiation of visceral adipocytes can be prepared by adding insulin and IGF-1 in the concentration ranges specified for the present invention to a basal cell culture medium. Any of the culture media typically used for cell culture (e.g., αMEM, F-12, SF02, PRMI1640, opti-MEM, and so forth) can be used as the basal cell culture medium. Preferably the serum used has been treated with heparin to remove the heparin-adsorbing components. Oils and fats may also be added to the culture medium of the present invention. The oil or fat used in the invention include, for example, plant oils and fats such as soy oil and coconut oil; animal oils and fats such as porcine tallow and beef tallow; and mineral oils such as paraffin. Oils and fats of plant origin are preferably used. A particularly preferred basal cell culture medium is the medium prepared by adding 17 mM pantothenic acid, 33 mM biotin, 100 mM ascorbic acid, 1 mM octanoic acid, 50 nM triiodothyronine, 100 units/mL penicillin, and 100 mg/mL streptomycin to (DMEM)/F12 containing 10% NCS (Shimizu, K., et al. Cell Biol. Int. XX (2006) 1-8).

The visceral preadipocytes can be obtained from tissues such as mesenteric fat, greater omental fat, periepididymal fat, and perirenal fat. For example, mesenteric fat is excised from the rat abdominal cavity, washed, minced with scissors, dispersed in buffer solution, and treated with collagenase. This is subjected to centrifugation and the floating cells are removed and the sediment is collected to yield the visceral preadipocytes. Differentiation into visceral adipocytes can be induced by seeding these precursor cells to the culture medium of the present invention (containing serum from which the heparin-adsorbing substances have been removed, as described above) in a cell culture vessel and incubating. The cell culture vessel may be, for example, an ordinary cell culture dish, flask or roller bottle. The cells may be cultured under usual culture conditions for mammalian cells, for example, at 37° C. under 5% CO₂. The culture medium may be changed once a day or once every several days. A primary culture of visceral adipocytes can be obtained in this manner.

Differentiation from the visceral preadipocytes the mature visceral adipocytes can be monitored by observing the presence of lipid droplets by visual or microscopic observation of the cells. Monitoring can also be carried out by, for example, quantitating the triglycerides in the cytoplasm, measuring the lipid synthesis activity (GPDH), measuring the amount of PPARγ mRNA, and so forth. C/EBPα participates in the acquisition of insulin sensitivity and is essential for differentiation into a adipocyte. It is also used as a marker of adipocyte differentiation. C/EBPβ induces C/EBPα and PPARγ gene expression in the early phase of the differentiation process. C/EBPβ itself undergoes transient expression, and, due to its reduced level of expression in the mature adipocyte, it is used as a marker of an undifferentiated or immature adipocyte. aP2 participates in fatty acid transport; its expression increases accompanying differentiation into the adipocyte and it is used as a adipocyte differentiation marker in the same manner as C/EBPα. Methods of measuring these markers have been established in the art.

The contents of all of the patents and references explicitly cited in this application are hereby incorporated by reference in its entirety. In addition, the content described in the specification and figures of Japanese Patent Application 2006-119749, which is the basis for the priority claiming by the instant application, is hereby incorporated by reference in its entirety.

### EXAMPLES

The present invention is described in greater detail by the examples provided below, but the present invention is not limited by these examples. The following abbreviations are used in the examples: VSC (visceral stromal cells=preadipocytes), VAC (visceral adipocytes=mature adipocytes), (insulin-like growth factor-1), C/EBPα (CCAAT enhancer binding protein α), C/EBPβ (CCAAT enhancer binding protein β), aP2 (adipocyte lipid-binding protein), IR (insulin receptor).

### Example 1

### Preparation of Preadipocytes and Medium

Mesenteric fat tissue was excised from the abdominal cavity of 3-5 week old male Sprague-Dawley rats (average body weight=100 g) and was washed three times with Hanks' balanced salt solution. The fat was minced with scissors and then incubated for 40 minutes at 37° C. in DMEM/F-12 culture medium containing 0.2% collagenase and 1.0% bovine albumin. The collagenase-digested tissue suspension was subsequently filtered through 600 μm mesh and Hanks' balanced salt solution was added. After centrifugation for 10 minutes at 800×g, the sediment was washed three times with Hanks' balanced salt solution and once with DMEM/F-12 culture medium and was then filtered through 100 μm mesh.

The cell suspension thus prepared from the intraabdominal mesenteric fat was seeded to dishes at 0.5×10⁵ cells/cm², and incubated at 37° C. and 5% CO₂ in the culture medium with the indicated composition. The culture medium was changed once a day or once every two days.

The culture medium was prepared by the addition of 17 mM pantothenic acid, 33 mM biotin, 100 mM ascorbic acid, 1 mM octanoic acid, 50 nM triiodothyronine, 100 unit/mL penicillin, and 100 mg/mL streptomycin to a basal culture medium of (DMEM)/F12 containing 10% NCS. Insulin and IGF-1 were added in the specified concentrations, and the resulting culture medium was used in the experiments described below.

### Example 2

### Effect of Dexamethasone on VAC Function

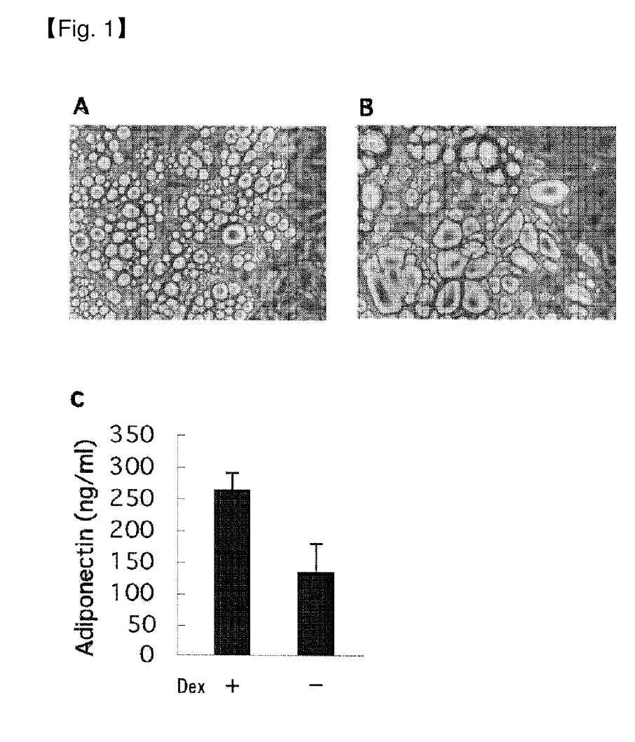

The conventionally used dexamethasone-based differentiation induction system was first evaluated. 2.5 μM dexamethasone and 10 μg/mL insulin or 10 μg/mL insulin was added to the medium to allow for VSC differentiation and maturation for 7 days. As shown in FIG. 1A, a normal fat accumulation occurs in the differentiation induction system containing insulin at a high concentration (10 μg/mL). When dexamethasone was also added to the differentiation induction culture medium, single cell enlargement was promoted (FIG. 1B), although there was no difference in the differentiation efficiency. In addition, the norepinephrine-induced triglyceride degradation reaction was reduced (data not shown). Moreover, when the amount of adiponectin present in the culture medium was measured after culture for seven days, a clear decline due to dexamethasone application was seen (FIG. 1C). These results demonstrate that adipocytes induced for differentiation by dexamethasone exhibit a reduction in their physiological function.

### Example 3

### Investigation of the Effect of Insulin on the Induction of VSC-to-VAC Differentiation

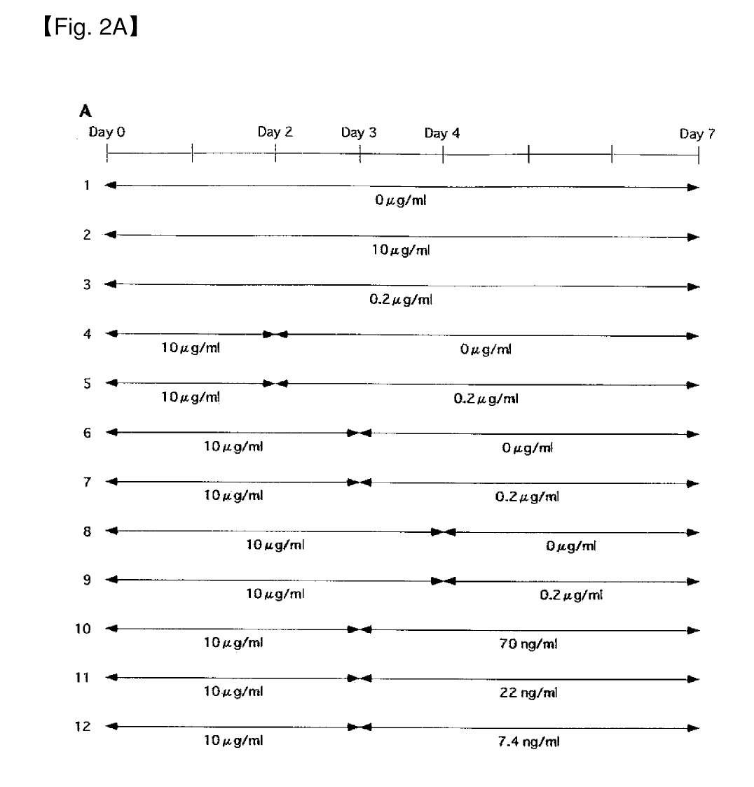

The influence of insulin on VSC-to-VAC differentiation was investigated using the various conditions shown in FIG. 2A. Differentiation conditions 1 to 12 in FIG. 2A correspond to the 1 to 12 in FIGS. 2B, 2C, and 2D (B: photographs after culture for 7 days; C: mRNA was separated after culture for 7 days, and the level of C/EBPα mRNA expression was measured by real-time PCR (the value on day 0 of culture was set at 1); D: the level of aP2 mRNA expression was measured by real-time PCR (the value on day 7 of culture for condition 1 was set at 1)).

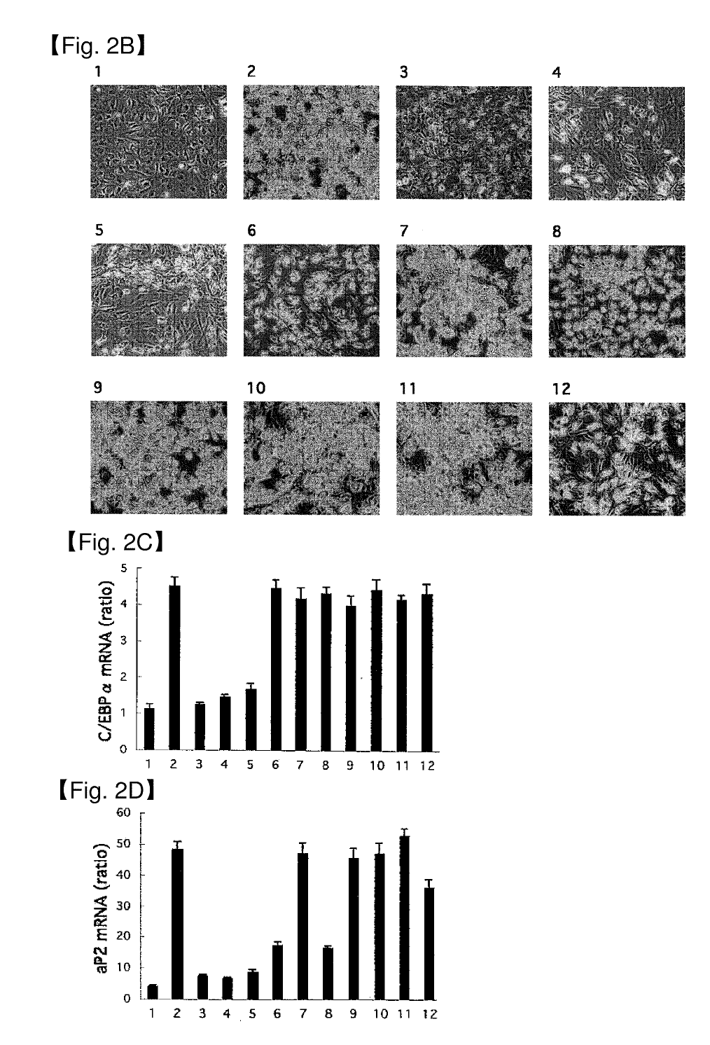

The accumulation of distinct lipid droplets was observed and differentiation into adipocytes occurred (FIG. 2B-2) with the medium containing high concentration insulin (10 μg/mL, condition 2) at a concentration several thousand times the physiological concentration, while differentiation did not occur (FIG. 2B-1) when no insulin was added (condition 1). When the insulin concentration of this differentiation induction culture medium was 0.2 μg/mL or 1/50th (condition 3), lipid droplet formation was entirely absent, which showed that the presence of insulin in a high concentration of 10 μg/mL was necessary for the induction of differentiation into VACs (FIG. 2B-3).

The schedule for treatment with 10 μg/mL insulin was then investigated. When differentiation induction was pursued by culture for a total of 7 days with 10 μg/mL insulin for 2 days (condition 5), 3 days (condition 7), or 4 days (condition 9) followed in each case by 0.2 μg/mL insulin, a significant decline in lipid droplet accumulation was seen for treatment for 2 days with 10 μg/mL insulin, demonstrating that treatment for at least 3 days is necessary (FIG. 2B-7). When a 3-day treatment with 10 μg/mL insulin was similarly carried out without the ensuing 0.2 μg/mL insulin, a significant decline in lipid droplet accumulation occurred in comparison to the system containing 0.2 μg/mL insulin. These results demonstrated that insulin is also required in the maturation process during lipid droplet formation after the induction of initial differentiation (FIGS. 2B-4, 2B-6, 2B-8). It has been reported that lipid droplet formation is seen even without insulin addition when immortalized 3T3-L1 adipocyte precursor cells are induced to differentiate into adipocytes (Entingh-Pearsall, A., and Kahn, C. R. (2004) J. Biol. Chem. 279 (36), 38016-38024. Epub 32004 Jul. 38017). Unlike the case for 3T3-LI adipocyte precursor cells, it was shown here that insulin is required for VSC-to-VAC differentiation. For the insulin-mediated induction of differentiation into adipocytes, it was found that differentiation induction by 10 μg/mL insulin for 3 days is required, and that subsequent maturation process is achieved by 0.2 μg/mL insulin.

Then the insulin concentration required in the adipocyte maturation phase after the induction of VSC-to-VAC differentiation was tested. Induction of VSC differentiation by 10 μg/mL insulin for 3 days was followed by treatment with insulin at a concentration of 10 μg/mL, 0.2 μg/mL, 70 ng/mL, 22 ng/mL, or 7.4 ng/mL (conditions 2, 7, 10, 11, and 12). The same level of lipid droplet formation was observed even when the insulin was reduced to 22 ng/mL (FIG. 2B-11). The insulin concentration of 22 ng/mL was about 10 times the physiological concentration.

The level of mRNA expression was also investigated (FIGS. 2C and 2D) for the adipocyte specific differentiation markers C/EBPα (adipocyte differentiation marker) and aP2 (adipocyte differentiation marker) for the treatment conditions with different insulin concentrations as shown in FIG. 2A. The C/EBPα mRNA expression level did not increase for the absence of insulin (condition 1), for the constant presence of 0.2 μg/mL insulin (condition 3), or for the presence of 10 μg/mL insulin for 2 days (conditions 4 and 5). The level of C/EBPα mRNA expression did rise when 10 μg/mL was added for only 3 days followed by the absence of insulin for 4 days (FIG. 2C-6). With regard to the level of aP2 mRNA expression, it was found that, compared to the constant presence of 10 μg/mL insulin (condition 2), less aP2 expression was observed in the absence of insulin (condition 1), in the case of the constant presence of 0.2 μg/mL insulin (condition 2), in the case of the presence of 10 μg/mL insulin for only 2 days (condition 5), and in the case of the maintenance of the insulin at or below 7.4 ng/mL after the presence of insulin at 10 μg/mL for 3 days (condition 12). In contrast, the presence of 10 μg/mL insulin for 3 days and maintenance of at least 22 ng/mL insulin (condition 11) showed the same level of aP2 mRNA expression as the constant presence of 10 μg/mL insulin (condition 2) (FIG. 2D). Accordingly, the results of the investigation of these differentiation markers also demonstrated that 10 μg/mL insulin is required only during the first 3 days of differentiation induction and thereafter an insulin concentration of 22 ng/mL is required.

The level of adiponectin mRNA expression was then examined with the aim of investigating the influence of the insulin concentration on VAC function (FIG. 3). After the VSCs had been differentiated by 10 μg/mL insulin for 3 days, they were then cultured for 4 days in the presence of 10 μg/mL insulin (lane 2), 0.2 μg/mL insulin (lane 3), 70 ng/mL insulin (lane 4), 22 ng/mL insulin (lane 5), or 7.4 ng/mL insulin (lane 6). After the 7-day culture, mRNA was separated and the amount of adiponectin mRNA expression was measured by real-time PCR. The value for the 7-day culture in the absence of insulin was set at 1. The results are shown in FIG. 3 (1: cultured for 7 days without insulin. 2: cultured for 7 days with 10 μg/mL insulin. 3: induction of differentiation for 3 days with 10 μg/mL insulin followed by culture for 4 days with 0.2 μg/mL insulin. 4: induction of differentiation for 3 days with 10 μg/mL insulin followed by culture for 4 days with 70 ng/mL insulin. 5: induction of differentiation for 3 days with 10 μg/mL insulin followed by culture for 4 days with 22 ng/mL insulin. 6: induction of differentiation for 3 days with 10 μg/mL insulin followed by culture for 4 days with 7.4 ng/mL insulin.).

The 7-day culture with 10 μg/mL insulin (lane 2) showed a lower level of adiponectin mRNA expression than the case of differentiation with 10 μg/mL insulin for 3 days followed by culture for 4 days with 0.2 μg/mL to 22 ng/mL (lanes 3 to 5). In addition, the level of adiponectin mRNA expression was reduced for 7.4 ng/mL insulin (lane 6).

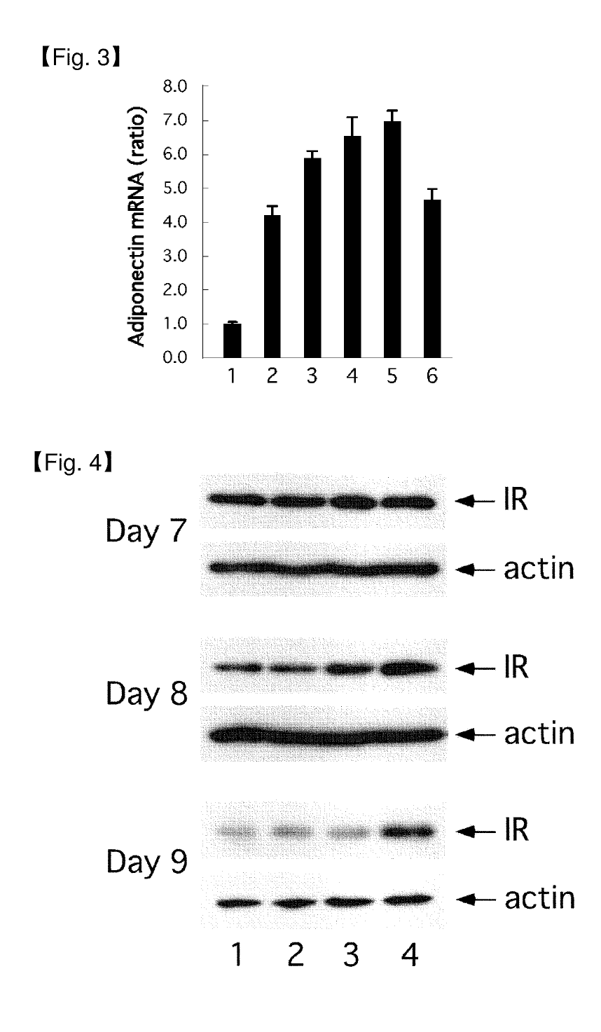

The amount of insulin receptor expression in the maturation process was then examined (FIG. 4). After the VSCs had been cultured for 7, 8, or 9 days, the total cell lysate (20 μg protein) was separated by SDS-PAGE and the IR was detected by immunoblotting with anti-IR antibody. In addition, actin was detected by immunoblotting with anti-actin antibody as the loading control. The results are shown in FIG. 4 (1: induction of differentiation for 3 days with 10 μg/mL insulin; then maturation for 2 days with 22 ng/mL insulin; then culture for the remaining time with 10 μg/mL insulin. 2: induction of differentiation for 3 days with 10 μg/mL insulin; then maturation for 2 days with 22 ng/mL insulin; then culture for the remaining time with 0.2 μg/mL insulin. 3: induction of differentiation for 3 days with 10 μg/mL insulin; then maturation for 2 days with 22 ng/mL insulin; then culture for the remaining time with 70 ng/mL insulin. 4: induction of differentiation for 3 days with 10 μg/mL insulin; then maturation for 2 days with 22 ng/mL insulin; then culture for the remaining time with 22 ng/mL insulin).

After VSCs were differentiated with insulin at 10 μg/mL for 3 days, followed by maturation with 22 ng/mL insulin, they were cultured with 10 μg/mL insulin (lane 1), 0.2 μg/mL insulin (lane 2), 70 ng/mL insulin (lane 3), or 22 ng/mL insulin (lane 4). Culture with insulin at 70 ng/mL or greater presented a substantial decline in insulin receptor expression from that for culture with insulin at 22 ng/mL (FIG. 4). These results showed that the amount of insulin receptor expression is reduced by treatment with high-concentration insulin after lipid droplet formation.

Conclusions from the preceding results are as follows. 1) The induction of VSC differentiation in the first 3 days is achieved by 10 μg/mL insulin, but is not achieved by 0.2 μg/mL insulin. Given that an insulin concentration of 0.2 μg/mL is a concentration nearly 100 times higher than that of the physiological concentration, the presence of another physiological factor in the induction of initial VSC differentiation was strongly suggested. 2) It was found, on the other hand, that the VAC maturation process after the induction of initial VSC differentiation required insulin at approximately 10 times the physiological concentration. 3) It was found that, when a high insulin concentration was present after the induction of initial VSC differentiation, expression of the insulin receptor was reduced and insulin resistance was induced. A conclusion was therefore drawn that, in a process of differentiation into VAC and maturation, nonphysiological disturbances are produced in the cell in the conventional culture methods, in which 10 μg/mL insulin is invariably added. At the same time, considering the fact that insulin in at least 100 times the in vivo level is required for the induction of initial differentiation and insulin in at least 10 times the in vivo level is also required for the maturation process, the conclusion was drawn that insulin alone is not sufficient for differentiation into the VAC and that another differentiation-inducing factor may participate in vivo.

### Example 4

### Investigation of the Influence of IGF-1 on the Induction of VSC-to-VAC Differentiation

The influence of IGF-1 was investigated with the aim of identifying the physiological factors in the induction of initial VSC differentiation. First, the control cell was VAC subjected to induction of initial differentiation with 10 μg/mL insulin (confirmed in FIG. 2) followed by maturation with 22 ng/mL insulin.

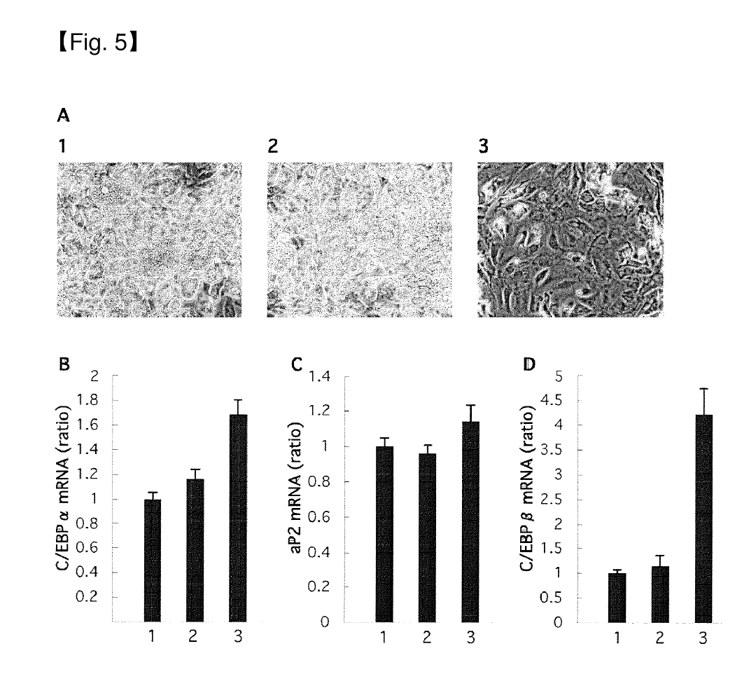

A continuous 7-day culture was carried out in a culture medium containing 200 ng/mL IGF-1 (normal concentration in human blood=150 to 250 ng/mL), and the amount of mRNA expression was then measured for C/EBPα (adipocyte differentiation marker), aP2 (adipocyte differentiation marker), and C/EBPβ (differentiation marker for undifferentiated adipocytes). The results are shown in FIG. 5 (A: photographs after culture for 7 days. 1: cultured with 200 ng/mL IGF-1 for 3 days and then with 22 ng/mL insulin for 4 days. 2: cultured with 200 ng/mL IGF-1 and 22 ng/mL insulin for 7 days. 3: cultured with 200 ng/mL IGF-1 for 7 days. The 1 to 3 corresponds to lanes 1 to 3 in FIGS. 5B to 5D. B: mRNA was separated after the 7-day culture and the amount of C/EBPα mRNA expression was measured by real-time PCR. The value for a culture of 3 days with 10 μg/mL insulin and then 4 days with 22 ng/mL insulin was set at 1. C: mRNA was separated after the 7-day culture and the amount of aP2 mRNA expression was measured by real-time PCR. The value for a culture of 3 days with 10 μg/mL insulin and then 4 days with 22 ng/mL insulin was set at 1. D: mRNA was separated after the 7-day culture and the amount of C/EBPβ mRNA expression was measured by real-time PCR. The value for a culture of 3 days with 10 μg/mL insulin and then 4 days with 22 ng/mL insulin was set at 1.).

The accumulation of lipid droplets was not seen for 200 ng/mL IGF-1 alone, and differentiation and maturation into VACs did not occur (FIG. 5A-3). This result was different from a previous report where lipid droplets are caused by IGF-1 alone (Boone, C., Gregoire, F., and Remacle, C. (2000) J. Anim. Sci. 78 (4), 885-895). On the other hand, the formation of lipid droplets to the same degree as the conventional culture methods was seen for the induction of differentiation by 200 ng/mL IGF-1 for 3 days followed by maturation for 4 days with 22 ng/mL insulin (FIG. 5A-1). In addition, the formation of lipid droplets to the same degree as the conventionally used culture methods was also seen for culturing for 7 days with 200 ng/mL IGF-1 and 22 ng/mL insulin (FIG. 5A-2). These results showed that initial differentiation is induced by a physiological concentration of IGF-1.

When the levels of C/EBPα and aP2 mRNA expression were investigated, the expression of C/EBPα and aP2 mRNAs was seen for the induction of differentiation by 200 ng/mL IGF-1 for 3 days followed by maturation for 4 days with 22 ng/mL insulin (FIGS. 5B and 5C). The level of expression was the same as the case where differentiation was induced with 10 μg/mL insulin followed by maturation for 4 days with 22 ng/mL insulin. The expression of C/EBPα and aP2 mRNAs was increased for the 7-day culture with only IGF-1 compared to that for induction of differentiation with 10 μg/mL insulin followed by maturation for 4 days with 22 ng/mL insulin, suggesting that IGF-1 at a physiological concentration adequately induced the expression of transcription factors (FIG. 5B, FIG. 5C, and 11 in FIG. 2). However, the amount of C/EBPβ mRNA, whose expression level is reduced by maturation, was maintained with alone (FIG. 5D, lane 3), which was in conformity with the morphological results. Therefore, it was suggested that by itself plays a sufficient role in the induction of initial differentiation, but that both IGF-1 and insulin may be required in the maturation process.

### Example 5

### Induction of VSC-to-VAC Differentiation by Combinations of and Insulin

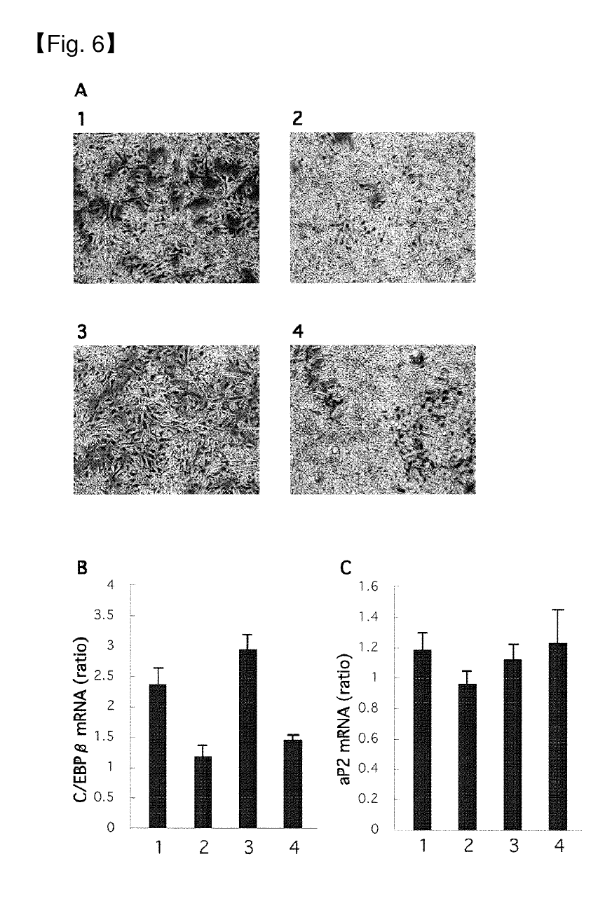

The influence of various combinations of IGF-1 and insulin on VAC differentiation induction and maturation was investigated. The results are shown in FIG. 6 (A: photographs after culture for 7 days. 1: cultured with 200 ng/mL IGF-1 for 3 days and then with 2.5 ng/mL insulin for 4 days. 2: cultured with 200 ng/mL IGF-1 and 2.5 ng/mL insulin for 7 days. 3: cultured with 200 ng/mL IGF-1 for 3 days and then with 0.85 ng/mL insulin for 4 days. 4: cultured with 200 ng/mL IGF-1 and 0.85 ng/mL insulin for 7 days. B: mRNA was separated after the 7-day culture and the amount of C/EBPβ mRNA expression was measured by real-time PCR. The value for a culture of 3 days with 10 μg/mL insulin and then 4 days with 22 ng/mL insulin was set at 1. C: mRNA was separated after the 7-day culture and the amount of aP2 mRNA expression was measured by real-time PCR. The value for a culture of 3 days with 10 μg/mL insulin and then 4 days with 22 ng/mL insulin was set at 1.).

Substantial lipid droplet formation was not observed when initial differentiation was induced for 3 days with 200 ng/mL IGF-1 followed by treatment for 4 days with 2.5 ng/mL (FIG. 6A-1) or 0.85 ng/mL (FIG. 6A-3) insulin, which are within the physiological concentration range. However, a substantial lipid droplet formation was seen for the 7-day cultures in the constant presence of 200 ng/mL IGF-1 plus 2.5 ng/mL (FIG. 6A-2) or 0.85 ng/mL (6A-4) insulin. These results showed that lipid droplet formation was not observed with IGF-1 alone but was promoted with insulin at a physiological concentration together with IGF-1. Accordingly, a successful differentiation and maturation into adipocytes is achieved with the combined presence of both IGF-1 and insulin within physiological concentration ranges.

Moreover, in agreement with the results in FIG. 5C, the expression of aP2 mRNA was increased in all cases, suggesting that a physiological concentration of IGF-1 adequately induces expression of transcription factors (FIG. 6C). However, the amount of C/EBPβ mRNA, whose expression level is reduced by maturation, was maintained in the case of culture with 200 ng/mL IGF-1 for 3 days followed by with 2.5 ng/mL or 0.85 ng/mL insulin for 4 days, while the amount of C/EBPβ mRNA was substantially reduced in the case of culture with 200 ng/mL IGF-1 plus 2.5 ng/mL or 0.85 ng/mL insulin for 7 days. (FIG. 5D, lane 3). These results suggested the possibility that a substantial induction of adipocyte differentiation could be brought about at physiological concentrations of insulin in the presence of both IGF-1 and insulin.

Additional investigations were then carried out into the IGF-1 concentration required for differentiation. Proceeding in the same manner as described above, VSCs were cultured for 7 days in the presence of 0.85 ng/mL insulin and IGF-1 at different concentrations. After culturing, mRNA was separated and the amount of C/EBPα mRNA expression was measured by real-time PCR. After culture for 7 days, mRNA was separated and the amount of C/EBPα mRNA expression and the amount of C/EBPβ mRNA expression were measured by real-time PCR. The value for the amount of mRNA expression for culture with 10 μg/mL insulin for 3 days followed by with 22 ng/mL insulin for 4 days was set at 1.

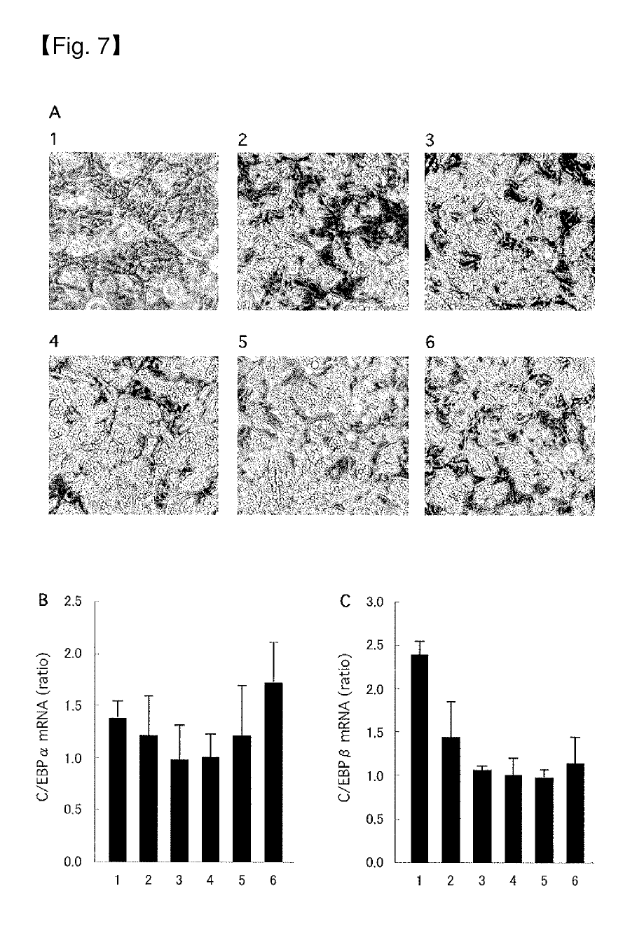

Photographs after culture for 7 days are shown in FIG. 7A; the amounts of C/EBPα mRNA expression are shown in FIG. 7B; and the amounts of C/EBPβ mRNA expression are shown in FIG. 7C. The 1 to 6 in FIG. 7A correspond to lanes 1 to 6 in FIGS. 7B and 7C.

1: 7-day culture with 10 ng/mL IGF-1 plus 0.85 ng/mL insulin.
2: 7-day culture with 50 ng/mL IGF-1 plus 0.85 ng/mL insulin.
3: 7-day culture with 100 ng/mL IGF-1 plus 0.85 ng/mL insulin.
4: 7-day culture with 200 ng/mL IGF-1 plus 0.85 ng/mL insulin.
5: 7-day culture with 400 ng/mL IGF-1 plus 0.85 ng/mL insulin.
6: 7-day culture with 800 ng/mL IGF-1 plus 0.85 ng/mL insulin.

A decline in the differentiation efficiency was observed at IGF-1 concentrations of 50 ng/mL and below. With respect to the amount of C/EBPα mRNA expression, no changes were seen as a function of the IGF-1 concentration; with respect to the C/EBPβ mRNA, IGF-1 concentrations of 50 ng/mL or less gave increased amounts of expression when compared with the presence of 200 ng/mL IGF-1, which was in agreement with the differentiation rates seen visually.

### Example 6

### Comparison With Differentiation Induction by High Insulin Concentrations

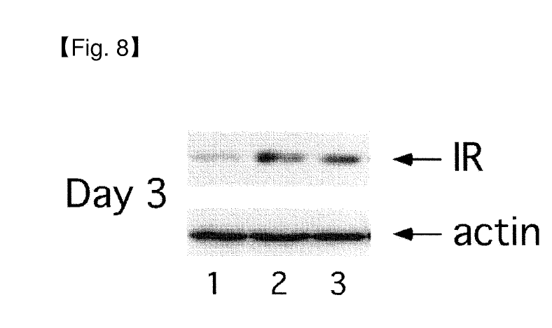

VSCs were cultured for 3 days in the presence of 10 μg/mL insulin or 200 ng/mL IGF-1, after which the total cell lysate (20 μg protein) was separated by SDS-PAGE and the insulin receptor was detected by immunoblotting with anti-insulin receptor (IR) antibody. Actin was detected by immunoblotting with anti-actin antibody as a loading control. The results are shown in FIG. 8 (1: with 10 μg/mL insulin for 3 days. 2: with 200 ng/mL IGF-1 for 3 days. 3: with 200 ng/mL IGF-1 and 22 ng/mL insulin for 3 days).

The amount of insulin receptor expression was compared after 3-day induction of initial differentiation by 10 μg/mL insulin or 200 ng/mL IGF-1. A decline in the insulin receptor was seen for the induction of differentiation by 10 μg/mL insulin in comparison to the induction of differentiation by 200 ng/mL IGF-1 (lane 2) and the induction of differentiation by 200 ng/mL IGF-1 and 22 ng/mL insulin (lane 3). The conclusion is therefore drawn that the adipocyte induced for differentiation with 10 μg/mL insulin does not adequately reflect the phenotypes of a normal adipocyte from the standpoint of research into the differentiation process.

### Example 7

### Hyperinsulinemia Model System

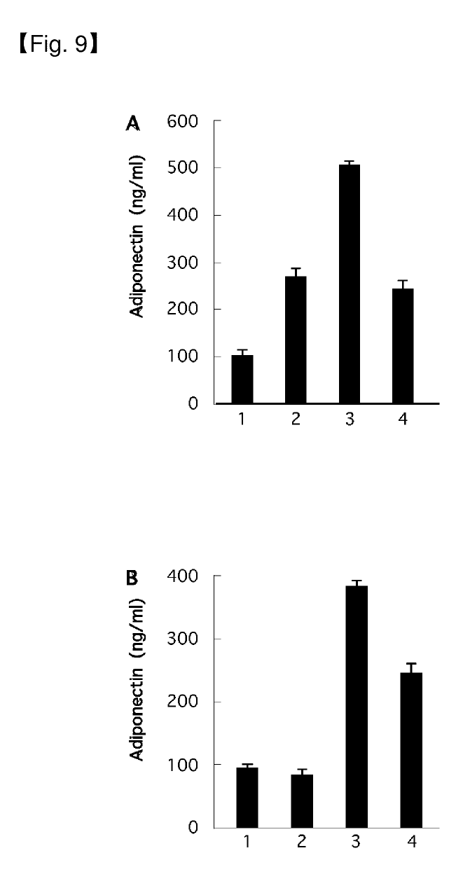

After 9-day culture with 200 ng/mL IGF-1 and different insulin concentrations, the culture medium was taken and the adiponectin content was measured by ELISA. When VSCs were cultured for 9 days with the constant presence of 200 ng/mL IGF-1 and insulin at a concentration of 10 μg/mL (lane 1), 70 ng/mL (lane 2), 22 ng/mL (lane 3), or 2.5 ng/mL (lane 4), the amount of adiponectin secretion was reduced at insulin concentrations of 70 ng/mL and above (FIG. 9A). When VSCs were cultured for the first 4 days with 200 ng/mL IGF-1 and 2.5 ng/mL insulin followed by 5 days with 200 ng/mL IGF-1 and insulin at a concentration of 10 μg/mL (lane 1), 70 ng/mL (lane 2), 22 ng/mL (lane 3), or 2.5 ng/mL (lane 4), adiponectin secretion was again reduced at insulin concentrations of 70 ng/mL and above (FIG. 9B). There results demonstrated that adiponectin secretion is inhibited by culture with insulin at 70 ng/mL or above and that adiponectin secretion is also inhibited by exposure to insulin at 70 ng/mL or above in the maturation process. An insulin concentration of 70 ng/mL is the level found in hyperinsulinemia patients. It was revealed from the above results that cultivation at an insulin concentration of 70 ng/mL of mature adipocytes obtained by the differentiation induction system of the present invention represents a hyperinsulinemia model system and can be used to screen and determine the efficacies of anti-hyperinsulinemic agents.

### Example 8

### Evaluation of Drug Candidates

Troglitazone is available on the market as an agent for ameliorating insulin resistance. An investigation was carried out into the effect of troglitazone on VACs which have been induced for differentiation in accordance with the present invention. VSCs were cultured for 7 days with 200 ng/mL IGF-1 and 0.85 ng/mL insulin; 10 μM troglitazone was added beginning on the fourth day of culture. After culture for 7 days, the total cell lysate (20 μg protein) was separated by SDS-PAGE and the IR was determined by immunoblotting with anti-IR antibody. Actin was detected by immunoblotting with anti-actin antibody as a loading control.

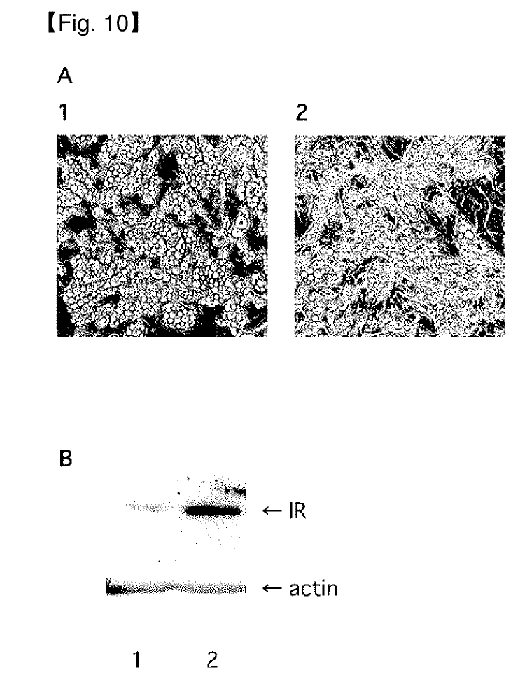

Photographs after culture for 7 days are given in FIG. 10A, while the amount of IR expression is shown in FIG. 10B. The symbols 1 and 2 in FIG. 10A correspond to lanes 1 and 2 in FIG. 10B.

1: cultured for 7 days with 200 ng/mL IGF-1 and 0.85 ng/mL insulin.
2: cultured for 7 days with 200 ng/mL IGF-1 and 0.85 ng/mL insulin; also with 10 μM troglitazone beginning on the fourth day of culture.

The results demonstrated that insulin receptor expression was increased by the addition of troglitazone, while no changes in morphology were observed.

### INDUSTRIAL APPLICABILITY

In accordance with the present invention, the differentiation of mesenteric adipocytes, which are intimately associated with lifestyle-related diseases such as diabetes, hyperlipidemia, arteriosclerosis, and so forth, can be induced under near-physiological conditions. The mature adipocytes obtained by the differentiation induction system of the present invention are therefore useful for elucidating the mechanisms underlying visceral fat accumulation and the onset of lifestyle-related diseases, and for evaluating and developing therapeutic agents and diagnostic reagents.

1. A culture medium for inducing the differentiation of visceral preadipocytes into mature visceral adipocytes comprising 0.85 to 30 ng/mL insulin and 50 to 250 ng/mL IGF-1 and is substantially free from indomethacin, dexamethasone, and IBMX.

2. (canceled)

3. The culture medium according to claim 1, comprising 0.85 to 5 ng/mL insulin and 50 to 250 ng/mL IGF-1.

4. A method of inducing the differentiation of visceral preadipocytes into mature visceral adipocytes, comprising culturing the visceral preadipocytes in the culture medium according to claim 1.

5. A method of assaying the efficacy of a test substance as an agent for ameliorating insulin resistance, comprising the steps of:
culturing mature visceral adipocytes obtained by induction using a culture medium containing 0.85 to 5 ng/mL insulin and 50 to 250 ng/mL IGF-1, under conditions that provoke insulin resistance to allow for inducing insulin resistance model visceral adipocytes; and
culturing said mature visceral adipocytes and said insulin resistance model visceral adipocytes in the presence of the test substance, and comparing the adipocyte function of these two types of cells.

6. A method of assaying the efficacy of a test substance as an agent for treating hyperinsulinemia, comprising the steps of:
comparing the level of insulin receptor expression between mature visceral adipocytes cultured in the presence of the test substance in a culture medium containing 0.85 to 5 ng/mL insulin and 50 to 250 ng/mL IGF-1, and mature visceral adipocytes cultured in the presence of the test substance in a culture medium containing 70 to 100 ng/mL insulin and 50 to 250 ng/mL IGF-1.

7. A method of assaying the efficacy of a test substance as an agent that stimulates the secretion of adiponectin, comprising the steps of:
culturing mature visceral adipocytes in the presence of the test substance in a culture medium containing 0.85 to 5 ng/mL insulin and 50 to 250 ng/mL IGF-1; and measuring the amount of adiponectin produced or secreted by the cells.

8. A method of assaying the efficacy of a test substance as an agent that stimulate the secretion of adiponectin in insulin resistance, comprising the steps of:
comparing the amount of adiponectin produced or secreted by mature visceral adipocytes cultured in the presence of the test substance in a culture medium containing 0.85 to 5 ng/mL insulin and 50 to 250 ng/mL IGF-1, and the amount of adiponectin produced or secreted by mature visceral adipocytes cultured in the presence of the test substance in a culture medium containing 70 to 100 ng/mL insulin and 50 to 250 ng/mL IGF-1.

9. A method of inducing the differentiation of visceral preadipocytes into mature visceral adipocytes, comprising culturing the visceral preadipocytes in the culture medium according to claim 3.
